(19) United States
(12) Patent Application Publication
(10) Pub. No.: US 2021/0369189 A1
(43) Pub. Date: Dec. 2, 2021

(54) BRUXISM DETECTION AND CORRECTION DEVICE

(71) Applicant: Olumide Bolarinwa, Mt. Airy, MD (US)

(72) Inventor: Olumide Bolarinwa, Mt. Airy, MD (US)

(21) Appl. No.: 17/330,954

(22) Filed: May 26, 2021

Related U.S. Application Data
(60) Provisional application No. 63/033,483, filed on Jun. 2, 2020.

(51) Int. Cl. A61B 5/00; A61B 5/08; A61B 5/15

(52) U.S. Cl. CPC A61B 5/4557; A61B 5/0826; A61B 5/681; A61B 5/6833; A61B 5/6815; A61B 2560/0475; A61B 5/150816; A61B 5/150809; A61B 5/0004; A61B 5/6898; A61B 2562/028; A61B 5/746

(57) ABSTRACT

The present invention is a small device containing at least the following elements: a MEMS microphone, an amplifier, a microprocessor with audio frequency filters, a wireless transmitter, and a battery. The device may be placed behind the ear of the user on a bony protrusion of the skull and may be held in place by a variety of known methods, including adhesives such as spirit gum, adhesive tape, and a small circular adhesive bandage. The MEMS microphone detects sounds transmitted through the skull by bone conductance and the microprocessor analyzes the sounds to determine whether they are associated with a bruxism event. If so, data associated with the sounds are sent via the wireless transmitter to an external device. The data may be stored for later review by the user or medical professional, or may trigger a response such as an auditory or vibratory alarm.

### BACKGROUND OF THE INVENTION

Bruxism, or the grinding or clenching of teeth, is a common condition affecting millions of people in the United States, alone; approximately 25% of all dental patients, by some estimates. Teeth grinding is commonly considered a behavior exhibited during sleep, but bruxism can also occur while awake, either as an unconscious habit or due to stress or anger. The intense physical stress induced during bruxism can cause a variety of adverse conditions, including, but not limited to: worn tooth enamel; fractured, chipped, or loose teeth; tooth pain and sensitivity; muscle soreness; sleep disruption; migraines and headaches; and damage to the temporomandibular joint. During sleep, the sounds created by bruxism can be disruptive to others attempting to sleep nearby, as well. In all, bruxism can adversely affect quality of life in numerous ways.

For those who are unaware of their bruxism, it is often a dentist who first alerts them to the behavior, after seeing signs of tooth wear or other physical damage. Often, a night guard is recommended to the patient. A night guard is a sturdy structure that sits over the top or bottom teeth, usually the top teeth. Night guards, also known as occlusal guards, may be fabricated as hard or soft materials, or a laminate of hard and soft materials. While these devices are effective at preventing damage to teeth, they do not address the underlying clenching behavior. Accordingly, night guards do not resolve other harmful aspects of bruxism, such as muscle soreness, sleep disruption, migraines and headaches, or damage to the temporomandibular joint. Furthermore, they are bulky to wear, which may further interfere with sleep, they may result in drooling, and after a few days of use, they become unsightly and odor producing. Also, because the device sits between the teeth of the upper and lower jaws, it forces the jaw into a more open position, which may cause the user's mouth to open while asleep and may increase the frequency or intensity of snoring. Because these guards do not change the underlying clenching/grinding behavior, they wear out and have to be replaced. Insurance coverage for the devices and for repair of damage caused by bruxism varies greatly or may not be covered at all.

Because occlusal guards cover the teeth, they affect the patient's speech, may be unsightly, can harbor odor-producing bacteria in the mouth, and are generally uncomfortable to wear during waking hours. Accordingly, they are generally only worn during sleep, so they also do not address bruxism while awake.

Others have attempted to make devices to address the harm caused by bruxism. Most of these devices have involved some form of device that sits inside the mouth, thus suffering the same flaws as the basic occlusal night guard described above, namely that they are bulky, uncomfortable, odor producing, and unsuitable for daytime use. See, for example, U.S. Pat. Nos. 5,666,973; 5,921,240; 6,675,804; 8,074,659; 8,439,044; 9,681,978; and D869,658. Some devices attempt to address the underlying behavior by emitting a tone or vibration to alert the user to their teeth clenching. See, for example, U.S. Pat. Nos. 5,078,153; 5,586,562; 9,398,974; and 9,827,137. One device even detects clenching, sending a signal to a set of plungers that are thrust into the user's nostrils in an attempt to force the patient to open their mouth to breath; see U.S. Pat. No. 10,195,071.

Others have attempted to address bruxism through devices that measure electrical conductance on the skin, resulting from contraction of the muscles of mastication. See, for example, U.S. Pat. Nos. 6,270,466; 8,588,883; and 8,690,800. These devices tend to be bulky, uncomfortable to use while attempting to sleep, and are too conspicuous for use in public while awake. Still others have used chemical applications, such as a device that releases a relaxant to the jaw muscles or a device that releases an unpleasant tasting substance into the mouth when bruxism is detected. See U.S. Pat. Nos. 6,638,241 and 6,164,278, respectively.

There is a need for a non-invasive device placed outside of the mouth that will identify bruxism both while the user is asleep and while awake, that will provide feedback to the user to modify the user's behavior, that is small enough to be comfortable to wear for a long period of time, and that is inconspicuous enough to use in public while awake.

### SUMMARY OF THE INVENTION

The present invention overcomes all of the disadvantages of the known devices meant to address bruxism, described above. The present device is small enough to fit behind the user's ear and be concealed from observation from the front of the user, enabling it to be used in public while the user is awake. Since the present invention is placed outside of the mouth, it does not interfere with speech, it does not cause drooling, is not unsightly, and does not harbor odor-producing bacteria in the mouth. The small size of the present invention enables it to be comfortably worn for extended periods of time, whether the user is awake or asleep. Further, the present invention is designed to address the behavior of bruxism and, therefore, to alleviate not only the physical damage to teeth, but also the other effects, such as muscle soreness, sleep disruption, migraines and headaches, and damage to the temporomandibular joint.

The present invention is a small device containing at least the following elements: a MEMS microphone, an amplifier, a microprocessor with audio frequency filters, a wireless transmitter, and a battery. Either rechargeable or non-rechargeable batteries are suitable for the invention. In a preferred embodiment, the invention contains a System-on-a-Chip (SoC) integrated circuit. The device may be placed behind the ear of the user on a bony protrusion of the skull, such as the mastoid process or the posterior portion of the zygomatic process. The device may be held in place by a variety of known methods, including adhesives such as spirit gum, adhesive tape, and a small circular adhesive bandage. The MEMS microphone detects sounds transmitted through the skull by bone conductance and the microprocessor analyzes the sounds to determine whether they are associated with a bruxism event. If so, data associated with the sounds, including but not limited to intensity, duration, and time of day are sent via the wireless transmitter to an external device, such as a smart phone, smart watch, smart alarm clock, a tablet device, or a laptop or desktop computer. Alternatively, the external device may be a stand-alone unit dedicated solely to the purposes of the invention.

Data associated with the bruxism event may simply be stored for later viewing by the user, or it may be used to trigger a response from the external device, such as an audible or vibratory alarm. The alarm may be configured to begin with a low intensity that gradually increases in intensity until the data from the device of the present invention indicates that bruxism has ceased. In this way, the device of the present invention may be used to train the user to stop bruxing with the least possible intrusion, whether the user is asleep or awake.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a lateral view of the human skull. The mastoid process and posterior portion of the zygomatic process are indicated as potential locations for the claimed invention.

FIG. 2 shows sound wave files comparing the sounds of chewing, breathing, speaking, and grinding teeth as measured through bone conductance collected at the mastoid process.

FIG. 3 shows sound wave files comparing the sounds of chewing, breathing, speaking, and grinding teeth as measured through bone conductance collected at the temporal bone.

FIG. 4 shows a flow chart diagram of the collection and processing of data from the claimed invention and transmission of processed data to an external device.

### DETAILED DESCRIPTION OF THE INVENTION

Reference is made herein to the attached drawings. Like reference numerals are used throughout the drawings to depict like or similar elements of the invention. For the purposes of presenting a brief and clear description of the present invention, a preferred embodiment will be discussed as used for reducing instances of bruxism. The figures are intended for representative purposes only and should not be considered to be limiting in any respect.

In physics, sound is a vibration that propagates as an acoustic wave, through a transmission medium such as a gas, liquid or solid. Sound can propagate through a medium such as air, water and solids as longitudinal waves and also as a transverse wave in solids.

In normal hearing, sound travels in waves through the air into the ear and ultimately reaches the eardrum. On the other side of the eardrum, three small bones transmit the vibration to the cochlea, which converts the sound waves into electrical impulses that are sent along the auditory nerve to the brain. But sound waves can also be transmitted through bones in the head. When these bones vibrate, the sound also reaches the cochlea, just as it would by going through the ear and eardrum, and results in the same sort of nerve impulses being transmitted to your brain. This method of sound transmission is called bone conduction.

The sound waves are generated by a sound source, such as in the present invention by the grinding of teeth. The sound source creates vibrations in the surrounding medium such as bone. As the source continues to vibrate the medium, the vibrations propagate away from the source at the speed of sound, thus forming the sound wave that propagates throughout the jaw bone and adjoining bone structures of the skull.

The earliest known application of bone conduction was by physician, mathematician, and philosopher Girolamo Cardano in his 1550 book, “De Subtilitate.” He found that he was able to hear sounds conducted through a rod or spear when placed between the teeth. More famously, Ludwig van Beethoven, the 18th century composer who was almost completely deaf, used a similar method, attaching a rod to his piano and clenching it in his teeth. He received perception of the sound when vibrations transferred from the piano to his jaw.

More recently, others have found practical applications for bone conduction. For example, in 1994 H. Werner Bottesch received U.S. Pat. No. 5,323,468 for a set of stereo music headphones designed for bone conduction. His device attached just behind the user's outer ears, so that it transmitted sound through the mastoid processes of the user's skull.

Thus, it is well known in the prior art that sound propagates through bone and that bone conduction can be used to bypass the outer and middle ear to convey externally generated ambient sound waves to the cochlea where ultimately hearing sensation is encoded as nerve electrical impulses transmitted to the brain.

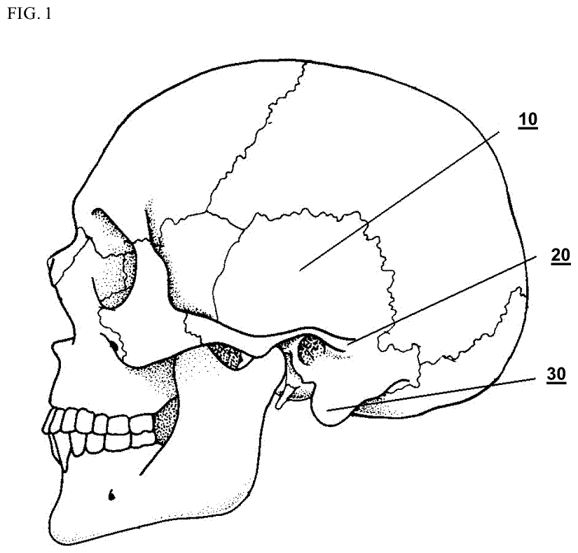

In the present invention, bone conduction is used to do the opposite, conveying internally generated sound (teeth grinding) to a small microphone placed over a bony structure external to skin. Referring now to FIG. 1, there is shown a lateral view of the human skull. Two locations on the temporal bone 10 are indicated: the mastoid process 30 and the posterior portion of the zygomatic process 20, as potential locations for the claimed invention. Because the invention works through collection of sound transmitted through the skull by bone conductance, one of ordinary skill in the art would recognize that the claimed invention may be placed on any portion of the skull where there is little muscle or fat tissue separating the skin from the bone, such as on the chin, the cheek, the forehead, etc. The locations 20 and 30 indicated in FIG. 1 have the added advantage of allowing the claimed invention to be easily concealed behind the user's ear and therefore less noticeable to others when used in public.

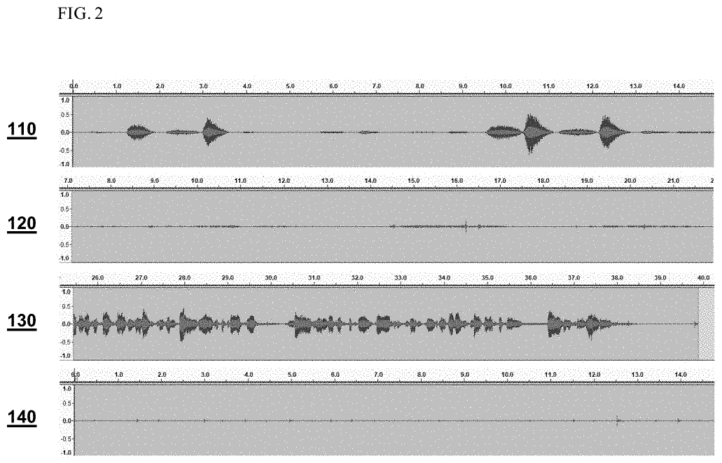

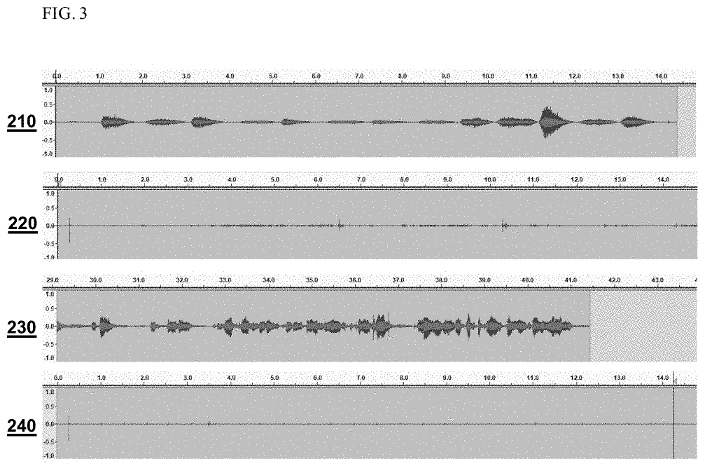

There are many sounds throughout the day originating within the oral cavity, including, but not limited to: chewing, swallowing, breathing, talking, coughing, snoring, teeth grinding or clenching, etc. Each of these sounds, however, generates a different type of sound wave. Sound waves are often simplified to a description in terms of sinusoidal plane waves, which can be characterized by these generic properties: frequency, wavelength, amplitude, and direction. Referring now to FIG. 2, there are shown a series of sound waves comparing the sounds of breathing 110, chewing 120, speaking 130, and grinding teeth 140 as measured through bone conductance collected at the mastoid process. Referring to FIG. 3, similar sound waves are shown, comparing the sounds of breathing 210, chewing 220, speaking 230, and grinding teeth 240 as measured through bone conductance collected at the temporal bone. These waves demonstrate that the various sounds originating in the oral cavity are readily distinguishable by their sound waves without ambiguity. FIGS. 2 and 3 also show that acoustic conduction of the distinct teeth grinding sound signature can be detected at both the mastoid process and the temporal bone from a simple surface microphone.

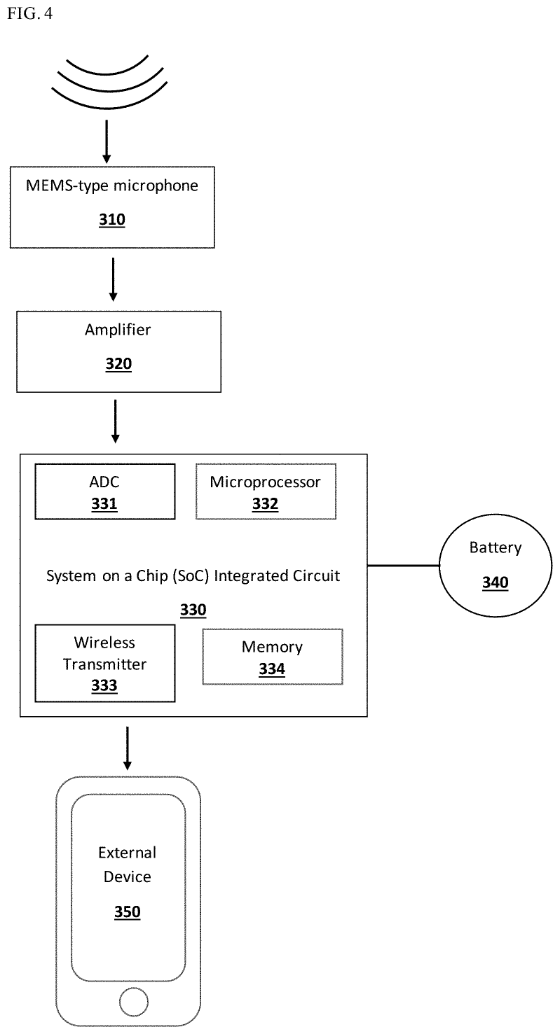

Referring now to FIG. 4, the system is comprised of four main components: a tiny MEMS-type microphone 310, an amplifier 320, a “system-on-a-chip” (SoC) integrated circuit 330, and a battery 340. The battery 340 may be a small, disposable button cell battery, such as those used in hearing aids, or may be a rechargeable battery. The SoC integrated circuit contains numerous elements, including, but not limited to an analog to digital converter (ADC) 331, a microprocessor 332, a wireless transmitter, such as a Bluetooth Low Energy transmitter 333, and a memory storage component 334.

The MEMS microphone 310 detects sound waves conducted through the bones of the skull and converts the vibrations to an electrical signal. The amplifier 320 amplifies the signal from the converted sound waves and sends the amplified signal to the ADC 331, which converts the signal to a digital form. The microprocessor 332 contains software or firmware to process the signal. The signal processing first evaluates the data stream and filters out signals outside the frequency range associated with teeth grinding. For example, sounds associated with breathing, talking, snoring, chewing, etc. may be removed. Optionally, an additional component may be disposed between the amplifier 320 and the SoC chip 330 to perform preliminary filtration of the data stream before it is converted to digital form aboard the SoC chip.

The filtered data stream is stored in memory 334 and then further filtered to eliminate electronic or other noise and to process the signal into a final state. The microprocessor then uses firmware to apply algorithms that confirm, characterize, and distinguish the signal profile as related to teeth grinding and the microprocessor then records the time and duration of the grinding activity and reports this data to an external device 350, such as a smart phone, smart watch, smart alarm clock, a tablet device, or a laptop or desktop computer. The user may be alerted by the external device through an audible or vibratory alarm, or just a data record showing time, date, and duration of teeth grinding activity. Optionally, the auditory or vibratory alarm may begin at a low intensity and gradually increase until the data transmitted by the claimed invention indicates that the bruxism event has ceased. In this way, the user may be alerted to the bruxism in the least intrusive manner possible. Ideally, during sleep, the level of feedback from the external device will be sufficient to stop the bruxing activity without waking the user entirely or unduly interfering with the user's sleep.

One of ordinary skill in the art would recognize that the device and process of the present invention could be applied to other activities other than bruxism. For example, the frequency filters and signal processing could be programmed to identify sounds associated with snoring and the external device could then alert the user to snoring behavior or simply log the time, duration, and intensity of snoring activity for later review by the user or transmission to an Ear Nose and Throat (ENT) physician for analysis.

The device of the present invention could also be modified to evaluate breathing patterns, airflow, and heart rate for analysis of sleep cycles and/or sleep apnea. An additional component could be included in the device to further analyze blood oxygen levels. These data points could then be logged and transmitted to a physician for analysis, reducing or even eliminating the need for expensive and time consuming in-hospital sleep studies.

The device of the present invention could be programmed to identify breathing sounds and log breathing patterns for analysis by a physician of various lung functions. This function could have applications relating to a variety of conditions including, asthma, sleep apnea, lung cancer, cystic fibrosis, or post-surgical or post-trauma lung function.

The device could also be used to measure the sound waves conducted through the bones of the skull that result from external physical contact. For example, the device could measure sounds associated with helmet-to-helmet contact in a football game to aid in the diagnosis of concussions or other traumatic injuries. Other components could be incorporated into the invention, such as one or more accelerometers to measure the severity of a helmet-to-helmet impact. The device could similarly be used to assess traumatic contact in other sports, such as baseball, basketball, racing, skiing, soccer, hockey, lacrosse, boxing, cycling, or motorcycling.

Incorporation of one or more accelerometers into the device of the present invention would also enable monitoring of ergonomic posture. The system could then be used, for example, to alert drivers if their posture suggests that they are losing alertness and are at risk of falling asleep at the wheel.

With respect to the above description, the optimum dimensional relationships for the parts of the invention, to include variations in size, materials, shape, form, function and manner of operation, assembly and use, are readily apparent and obvious to one of ordinary skill in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An apparatus comprising:
a MEMS microphone operative to detect sound waves transmitted by bone conductance through the skull of a user and to convert those sound waves to an electronic signal;
an amplifier operative to increase the amplitude of the electronic signal;
an analog to digital converter operative to convert the amplified signal into a digital form;
a microprocessor with software or firmware operative to process the digital signal;
a wireless transmitter operative to wirelessly send the processed signal to an external device;
and a battery.

2. The apparatus of claim 1, wherein the analog to digital converter, microprocessor, and wireless transmitter are housed within an integrated circuit.

3. The apparatus of claim 2, wherein the integrated circuit further comprises a memory storage device.

4. The apparatus of claim 1, wherein the wireless transmitter is a Bluetooth Low Energy transmitter.

5. The apparatus of claim 1, wherein the apparatus is small enough to be secured and concealed behind the ear of the user on a bony protrusion of the skull.

6. The apparatus of claim 5, wherein the means to secure the device are selected from an adhesive, adhesive tape, and an adhesive bandage.

7. The apparatus of claim 1, wherein the software or firmware is operative to analyze the digital signal, identify sounds associated with specific auditory events, and send only data relating to particular auditory events to the wireless transmitter.

8. The apparatus of claim 7, wherein the auditory events are selected from bruxism, snoring, breathing, and pulse.

9. The apparatus of claim 2, further comprising an analog data filter disposed between the amplifier and the integrated circuit.

10. The apparatus of claim 1, wherein the external device is selected from a smart phone a smart watch, a smart alarm, a tablet device, a laptop computer, and a desktop computer.

11. A method for detecting and correcting bruxism, said method comprising the steps of:
providing an apparatus as defined in claim 1, comprising a MEMS microphone, an amplifier, an analog to digital converter, a microprocessor, a memory storage device, a wireless transmitter, and a battery;
securing the apparatus to a bony protrusion of the skull, behind a user's ear using a means selected from an adhesive, adhesive tape, or an adhesive bandage;
detecting sounds transmitted by bone conduction through the user's skull with the MEMS microphone that converts those sounds to an analog electronic signal;
sending the analog signal to the amplifier, which increases the amplitude of the signal;
sending the amplified signal to the analog to digital converter, which converts the signal to digital form;
sending the digital signal to the microprocessor;
analyzing the digital signal with software or firmware in the microprocessor, said analysis comprising:
filtering frequencies outside the range associated with teeth grinding;
storing the filtered data the memory storage device;
further filtering the data stream to eliminate electronic or other noice;
processing the signal into a final state;
applying algorithms that confirm, characterize, and distinguish the signal profile as related to teeth grinding;
creating reporting data containing the time and duration of the grinding activity;
sending the reporting data to an external device selected from a smart phone, smart watch, smart alarm clock, tablet device, laptop computer, desktop computer, or dedicated receiving unit;
providing feedback to the user that a bruxism event has occurred.

12. The method of claim 11, wherein the external device stores the reporting data for later review by the user or a medical professional.

13. The method of claim 11, wherein the feedback is selected from an audible alarm, a vibratory alarm, or a combination thereof.

14. The method of claim 13, wherein the auditory or vibratory alarm begins at a low intensity and gradually increases until the reporting data transmitted by the apparatus to the external device indicates that the bruxism event has ceased.

15. The method of claim 11, wherein the external device transmits a signal to a second external device to initiate an audible alarm, vibratory alarm, or a combination thereof.

16. The method of claim 11, further comprising the step of passing the amplified data through an analog data filter prior to entering the analog to digital converter.

17. The apparatus of claim 1, further comprising a pulse oximeter.

18. The apparatus of claim 1, wherein the software or firmware adapted to identify and analyze data relating to blood oxygen levels and sounds associated with snoring, breathing patterns, airflow, and heart rate.
